(19) United States
(12) Patent Application Publication
(10) Pub. No.: US 2006/0078578 A1
(43) Pub. Date: Apr. 13, 2006

(54) Cosmetic compositions with montmorillonite stabilizing agent

(76) Inventors: Robert Walter Sandewicz, Monroe Township, NJ (US); Frank Charles Pagano, Monroe Township, NJ (US); Anjali Abhimanyu Patil, Westfield, NJ (US); Joseph Frank Calello, Bridgewater, NJ (US)

Correspondence Address: Julie Blackburn;Revlon Consumer Products Corporation, 237 Park Avenue, New York, NY, 10017, US

(21) Appl. No.: 10/960,557

(22) Filed: Oct. 7, 2004

(51) Int. Cl. A61K 8/26; A61K 8/25

(52) U.S. Cl. 424/401

(57) ABSTRACT

A stabilized film forming cosmetic composition comprising a stabilizing effective amount of dispersed non-quaternary montmorillonite mineral and a film forming effective amount of a film forming component, and a method for compatibilizing ingredients in long wearing or transfer resistant cosmetic compositions by formulating such compositions with a non-quaternary montmorillonite mineral.

### TECHNICAL FIELD

The invention is in the field of cosmetic compositions for application to keratinous surfaces such as skin, hair, or nails for the purpose of coloring, conditioning, treating, or otherwise beautifying the keratinous surface.

### BACKGROUND OF THE INVENTION

Cosmetic formulators are always looking for ways, to improve cosmetic formulas. Generally, there are both aesthetic and practical considerations. Aesthetic considerations include consumer perceivable properties such as wear, adhesion, gloss, comfort, application characteristics and the like. Practical considerations are of also of concern to the cosmetic manufacturer, and include properties such as formula stability, compatibility between formula and package, and other characteristics necessary to ensure that the product ultimately delivered to the consumer is commercially acceptable.

Cosmetic manufacturers are continually looking to improve the wear of cosmetic formulas. Today's women lead very busy lives and are not inclined to take time out from their activities to touch up or reapply makeup. Wear is often achieved by incorporating various film forming ingredients into cosmetic compositions. Such film forming ingredients cause the composition to form a film that holds the actives in the formula on the keratinous surface for a longer period of time. In some cases the film formers and other ingredients present are not particularly compatible. Often the various ingredients that provide the best cosmetic are not inherently compatible, which means that the cosmetic itself will never be commercially acceptable unless some ingredient or process is used to compatibilize the inherently incompatible ingredients.

Accordingly, when making cosmetic compositions that contain film forming properties there is a need for agents which will stabilize the formula or compatibilize the various ingredients in the formula so that they can be used together without concern for formula separation or incompatibility.

It is an object of the invention to provide stabilized cosmetic compositions containing a film forming component and an effective stabilizing agent that is a non-quaternary montmorillonite mineral.

It is also an object of the invention to provide stabilized transfer resistant and/or long wearing cosmetic compositions containing a film forming component and a non-quaternary montmorillonite mineral.

It is also an object of the invention to provide stabilized cosmetic compositions that are internally compatible.

### SUMMARY OF THE INVENTION

The invention is directed to a film forming cosmetic composition comprising a stabilizing effective amount of at least one dispersed non-quaternary montmorillonite mineral, and a film forming effective amount of at least one film forming component.

The invention is further directed to a cosmetic composition comprising at least one dispersed non-quaternary montmorillonite mineral in nanoplatelet form.

The invention is further directed to a method for compatibilizing ingredients in a transfer resistant and/or long wearing cosmetic composition containing at least one film forming component, comprising formulating said composition with at least one non-quaternary montmorillonite mineral.

### DETAILED DESCRIPTION

I. The Composition

A. The Montmorillonite Mineral

The composition of the invention comprises a stabilizing effective amount of at least one non-quaternary montmorillonite mineral. The term “stabilizing effective amount” means that the montmorillonite mineral is present in an amount sufficient to exert a stabilizing effect on the composition such that the composition containing the montmorillonite mineral is more stable than a composition that is the same or similar and does not contain the montmorillonite mineral. The term “stable” means that the composition is stable enough, and meets the general requirements necessary to provide a commercially acceptable product. Generally a stabilizing effective amount of the non-quaternary montmorillonite mineral may range from about 0.001-85%, preferably about 0.01-75%, more preferably about 0.1-60% by weight of the total composition. The montmorillonite mineral may be hydrated, although it may be used as effectively in the non-hydrated state.

The montmorillonite mineral is non-quaternary, which means that it has not been reacted with quaternary ammonium compounds, such as Quaternium-18 or distearyl dimonium chloride and the like. The montmorillonite mineral is generally in the form of platelets or sheets that may or may not be interconnected. In the most preferred embodiment of the invention, the montmorillonite mineral has been sheared to form nanoparticles or nanoplatelets, or sheets, of very small thickness. Preferably such nanoplatelets have thicknesses ranging from about 0.5 to 5 nanometers (0.0005 to 0.005 microns). Preferably, the top surface area of the nanoplatelet ranges from about 20 to 2000 nanometers (0.02 to 2 microns). In one of the most preferred embodiments of the invention the montmorillonite mineral is nonionic, although it may contain various groups such as anionic or cationic groups that will impart charge.

Suitable montmorillonite minerals include synthetic or natural metal silicates such sodium, potassium, magnesium, aluminum, lithium, zinc, iron, calcium, or beryllium silicates or mixtures thereof. Natural metal silicates are also known as “hectorites” or “bentonites”. In general, a formula for some types of clays in the montmorillonite group is as follows:
(Na, Ca)(Al, Mg)₆(Si₄O₁₀)₃(OH)₆₋ₓH₂O)

Where x is the variable amount of water that may be present.

Particularly preferred for use in the compositions of the invention are non-quaternary montmorillonite minerals purchased from Amcol Health and Beauty Solutions and sold under the Magnasperse™. These montmorillonite minerals may be solvated or dispersed in aqueous or non-aqueous solvents. Examples of suitable aqueous solvents include water, alkylene glycols, or mono-, di-, or polyhydric alcohols. Examples of suitable non-aqueous solvents include volatile cyclic or linear silicones, volatile paraffinic hydrocarbons, non-volatile organic oils or silicones, and the like.

B. Dispersion Enhancing Agents

The montmorillonite mineral alone may be dispersed in the cosmetic composition or, if desired, it may be used with one or more dispersion enhancing agents that facilitate better dispersion of the montmorillonite mineral in the composition. Generally, such dispersion enhancing agents act by separating the platelets when they are found in the “stack of cards” or closely layered formation. If present, such dispersing agents are preferably present in amounts ranging from about 0.0001 to 50%, preferably about 0.001-40%, more preferably about 0.01-35% by weight of the total composition. Particularly suitable dispersion enhancing agents are polar.

A variety of dispersion enhancing agents are suitable, including:

1. Solvents

Also suitable as dispersing agents are various types of solvents such as water, or ethanol, propanol, isopropanol, or propylene carbonate. Particularly preferred is propylene carbonate.

2. Polyesters. Polyethers, or Polyester/Polvethers

Also suitable as the dispersion enhancing agents are polyesters or polyethers, particularly those containing fatty groups. Suitable polyesters include polymeric fatty acids or hydroxy fatty acids such as poly(12-hydroxy)stearic acid, polyhydroxy stearic acid, where the number of repeating fatty acid units may range from about 2 to 500,000 units.

Also suitable are polyester/polyethers, which contain both repeating fatty acid and alkyl ether groups. Such ingredients are typically formed by reacting polyesters with polyethers. More preferred is a polyester/polyether where the polyester is a repeating fatty acid ester which may or may not be substituted with hydroxyl groups, and the polyether is a repeating alkyl ether such as a glycol. Particularly preferred is where the repeating fatty acid ester is a hydroxy fatty acid ester, particularly dihydroxy stearic acid or dihydroxy isostearic acid, and the polyether is a repeating ethylene or propylene glycol group. Most preferred is a polyester/polyether that is PEG-30 dipolyhydroxystearate.

C. Film Forming Component

The cosmetic compositions of the invention contain one or more film forming components. Suitable film forming components may include polymers, high molecular weight hydrocarbons or esters, waxes, and the like. Preferably the composition contains from about 0.01-95%, preferably from about 0.05-85%, more preferably from about 0.1-80% by weight of the total composition of film forming component. The different types of film forming components are further described herein:

1. Silicone Polymers

Suitable film forming components include one or more silicone polymers. The term “silicone polymer” or “siloxane polymer” means a polymer comprised of a backbone of repeating silicon and oxygen atoms, and which comprises one or more of monofunctional, difunctional, trifunctional or tetrafunctional siloxy units.

The term monofunctional means that the unit contains one oxygen atom that is shared by 2 silicon atoms when the monofunctional unit is polymerized with one or more of the other types of units. In silicone nomenclature used by those skilled in the art, a monofunctional siloxy unit is designated by the letter “M”, and means a unit having the general formula:
R₁R₂R₃SiO_{1/2}
wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula: 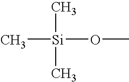
When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D” designation is sometimes used, which indicates a substituent other than methyl. For purposes of this disclosure, a “D” unit has the general formula:
R₁R₂SiO_{2/2}
wherein R₁ and R₂ are defined as above. The SiO_{2/2} designation means that the silicon atom in the difunctional unit is bonded to two oxygen atoms when the unit is copolymerized with one or more of the other units. For example, when R₁, R₂, are methyl the resulting difunctional unit is of the formula: 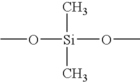
When this difunctional unit is polymerized with one or more of the other units the silicon atom will be bonded to two oxygen atoms, i.e. will share two halves of an oxygen atom.

The term “trifunctional siloxy unit” is generally designated by the letter “T” in standard silicone nomenclature. A “T” unit has the general formula:
R₁SiO_{3/2}
wherein R₁ is as defined above. The SiO_{3/2} designation means that the silicon atom is bonded to three oxygen atoms when the unit is copolymerized with one or more of the other units. For example when R₁ is methyl the resulting trifunctional unit is of the formula: 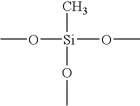
When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:
SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

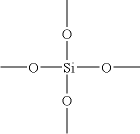

The film forming silicone polymer used in the compositions of the invention may be any combination of M, D, T, or Q units so long as the polymer is capable of forming a cosmetic film on a keratinous surface that includes skin, hair, or nails.

The silicone polymer may be a liquid, semi-solid, or solid at room temperature. It may be a gum or resin. The term “gum” generally means a high molecular weight silicone polymer that may be cross-linked or uncrosslinked, and preferably comprises M and D units, and possibly some T or Q units. Examples of suitable silicone gums include high molecular weight polydimethylsiloxanes having viscosities in the range of about 100,000 to 90,000,000, preferably about 500,000 to 10,000,000 centipoise at 25° C. In the case where the silicone gum is crosslinked, one or more of the M, D, or T units may be substituted with hydrogen atoms, which will react with suitable cross linking groups such as vinyl or alpha omega dienes, or possibly other reactive sites on substituted M, D, or T units. For example, cross-linking can occur when a polydimethyl-methylhydrogen siloxane is polymerized in the usual manner with a second polydimethylsiloxane that is substituted with one or more vinyl or alpha omega diene functional groups.

The silicone polymer used as the film forming component may be a silicone resin. The term “silicone resin” when used herein means a silicone containing T, MT, or MQ units. The term “T” means that the silicone comprises mostly T units, as above defined, either alone or in combination with D or M units. The term “MT” means that the silicone contains at least M and T units as defined above and possibly minor amounts of other types of units. The term “MQ” means that the silicone resin comprises at least M and Q units as defined above and possibly minor amounts of other types of units.

Typically T or MT silicones are referred to as silsesquioxanes, and in the case where M units are present methylsilsesquioxanes. Such silsesquioxanes have the general formula:
(R₁SiO_{3/2})x
where x ranges from about 1 to 100,000 and wherein R₁ is as defined above.

Certain types of such silsesquioxanes are manufactured by Wacker Chemie under the Resin MK designation. This particular type of polymethylsilsesquioxane is a polymer comprised of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polymethylsilsesquioxanes that may be used as the film forming component include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

Another particular type of silicone polymer that is suitable as a film forming component is an MQ resin, also generally referred to as a siloxy silicate resin. Such silicones generally have the formula:
[R₁R₂R₃SiO_{1/2}]ₓ[SiO_{4/2}]_{y}
wherein R₁, R₂, and R₃ are each independently as defined above, and, preferably, x and y are such that the ratio of [R₁R₂R₃SiO_{1/2}] to [SiO_{4/2}] units is 0.5 to 1 to 1.5 to 1. In this type of resin, preferably R₁, R₂, and R₃ are a C₁₋₆ alkyl, and more preferably are methyl and x and y are such that the ratio of [R₁R₂R₃SiO_{1/2}] to [SiO_{4/2}] units is 0.75 to 1. One type of preferred MQ resin is a trimethylsiloxy silicate containing 2.4 to 2.9 weight percent hydroxyl groups, which is formed by the reaction of the sodium salt of silicic acid, chlorotrimethylsilane, and isopropyl alcohol. The manufacture of such a trimethylsiloxy silicate is set forth in U.S. Pat. Nos. 2,676,182; 3,541,205; and 3,836,437, all of which are hereby incorporated by reference. Trimethylsiloxy silicate as described is also available from Dow Corning Corporation under the tradename Dow Corning 749 Fluid (former known as Dow Corning 2-0749 Fluid), which is a blend of about 40-60% volatile silicone and 40-60% trimethylsiloxy silicate. Dow Corning 749 Fluid in particular, is a fluid containing about 50% trimethylsiloxy silicate and about 50% cyclomethicone. The fluid has a viscosity of 200-700 centipoise at 25.degree. C., a specific gravity of 1.00 to 1.10 at 25.degree. C., and a refractive index of 1.40-1.41. Also suitable are MQ resins manufactured by GE Silicones under the tradename SR1000, and Wacker silicones under the tradename TMS 803.

Typically silicone polymers that may be used in the composition are made according to processes well known in the art. In general, siloxane polymers are obtained by hydrolysis of silane monomers, preferably chlorosilane monomers. The chlorosilanes are hydrolyzed to silanols and then condensed to form siloxanes. The hydrolysis and condensation may leave some residual hydroxy or alkoxy functionality on the siloxane. The silicone film forming polymers used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

Other suitable silicone polymers include dimethicones, which are linear silicones comprised of M and repeating D units.

2. Polymers of Siloxane Monomers and Organic Monomers

Also suitable as the film forming component are polymers obtained by polymerization of siloxane monomers and organic monomers such as ethylenically unsaturated monomers, urethanes, amides, or mixtures thereof. Preferably this type of film forming polymer comprises a copolymer of siloxane monomers and ethylenically unsaturated monomers.

The siloxane monomers in the polymer may be obtained by polymerization of any one or more of the following siloxane units mentioned in (1), above, with respect to the silicone polymer. Preferably, the siloxane portion of the copolymer is prepared by polymerizing the desired siloxane units to form the polymer chain. The siloxane chain or portion is then grafted or attached to the organic portion of the copolymer, which may already be polymerized into a polymer chain. Alternatively, the organic polymer is synthesized in situ in a reaction medium containing the siloxane chain. The siloxane chain itself may be a liquid, semi-solid, or solid at room temperature. It may be a gum or resin as defined in (1) above.

The siloxane chain for use in making the copolymers used in the compositions of the invention are made according to the processes set forth in (1), above.

The ethylenically unsaturated monomers that may be used to form the organic portion of the polymer may include repeating C₁₋₃₀ alkylenes such as ethylene, propylene, butylene, and the like; as well as acrylic acid, methacrylic acid, either alone or esterified with C₁₋₃₀ alkanols; styrene, styrene substituted with C₁₋₁₀ alkyl groups (e.g. t-butyl styrene), vinyl pyrrolidone, and the like. The various types of ethylenically unsaturated monomers include, but are not limited to, those of the general formula: 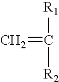
wherein R₁, and R₂ are each independently H, halogen, hydroxyl, fluoroalkyl, a C₁₋₃₀ straight or branched chain alkyl, aryl, aralkyl; R₂ is a pyrrolidone, or a substituted or unsubstituted aromatic, alicyclic, or bicyclic ring where the substituents are C₁₋₃₀ straight or branched chain alkyl, or COOM or OCOM herein M is a C₁₋₃₀ straight or branched chain alkyl, pyrrolidone, or a substituted or unsubstituted aromatic, alicylic, or bicyclic ring where the substituents are C₁₋₃₀ straight or branched chain alkyl.

Types of preferred monomers include styrene, or styrene substituted with methyl, ethyl, propyl, or butyl groups, either alone or in combination with other types of ethylenically unsaturated monomers such as acrylic acid, methacrylic acid, simple esters of acrylic acid, methacrylic acid, ethylene, propylene, butylene, and the like.

If desired, another type of monomer that may be polymerized with the siloxane monomers is a urethane monomer. Urethanes are generally formed by the reaction of polyhydroxy compounds with diisocyanates, as follows:

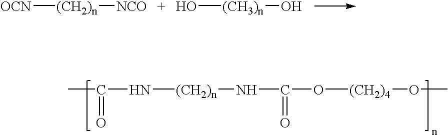

Another type of monomer that may be polymerized with the siloxane monomers comprises amide groups, preferably having the following formula: 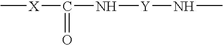
wherein X and Y are each independently linear or branched alkylene having ₁₋₄₀ carbon atoms, which may be substituted with one or more amide, hydrogen, alkyl, aryl, or halogen substituents. Examples of such amides and amide groups are set forth in U.S. Pat. No. 6,353,076, which is hereby incorporated by reference in its entirety.

More specific examples of suitable polymers include, but are not limited to the following:

| Siloxane Chain | |
|---|---|
| Selected From | Organic Chain Selected From |
| M, D, T, or Q | ethylene, butylene, styrene, butyl styrene, |
| | methyl styrene, acrylate, methacrylate, |
| | acrylic acid, methacrylic acid, butyl |
| | acrylate, butyl methacrylate, methyl |
| | methacrylate, propyl methacrylate, |
| | methyl acrylate, ethyl hexyl acrylate or |
| | methacrylate, ethyl acrylate or methacrylate, vinyl |
| | toluene, cyclic acrylates or methacrylates, isobornyl |
| | methacrylate, urethane, or mixtures thereof |

Further nonlimiting examples of other types of copolymers that may be used as the film forming component include: copolymers of trimethylsiloysilicate and butyl acrylate and butyl styrene; copolymers of trimethylsiloxysilicate and butyl styrene, copolymers of trimethylsiloxysilicate, butyl methacrylate, and butyl styrene, copolymers of trimethylsiloxysilicate, butyl methacrylate, and methyl styrene, copolymers of polymethylsilsesquioxane, butyl styrene, and methyl acrylate, copolymers of polymethylsilsesquioxane, methacrylic acid, and styrene, copolymers of polymethylsilsesquioxane and styrene, copolymers of dimethicone, butyl styrene, and methyl methacrylate, copolymers of dimethicone, styrene, and ethyl hexyl methacrylate, copolymers of dimethicone, butyl styrene, ethyl methacrylate, and so on. The variations may be unlimited and any one or more of the silicone types M, D, T, or Q, may be copolymerized with any one or more of the organic monomers mentioned herein.

3. Polymers from Ethylenically Unsaturated Monomers

Also suitable for use as the film forming component are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

Particular examples of such polymers include those containing one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups. Also suitable are methacrylic acid or acrylic acid esters esterified with moieties containing alicyclic or bicyclic rings such as cyclohexyl or isobomyl, for example.

The ethylenically unsaturated monomer may be mono-, di-, tri-, or polyfunctional as regards the addition polymerizable ethylenic bonds. A variety of ethylenically unsaturated monomers are suitable.

Examples of suitable monofunctional ethylenically unsaturated monomers include those of the formula: 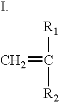
wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substituents are C₁₋₃₀ straight or branched chain alkyl, or COOM or OCOM wherein M is H, a C₁₋₃₀ straight or branched chain alkyl, pyrrolidone, or a substituted or unsubstituted aromatic, alicylic, or bicyclic ring where the substituents are C₁₋₃₀ straight or branched chain alkyl which may be substituted with one or more hydroxyl groups, or [(CH₂)ₘO]ₙH wherein m is 1-20, and n is 1-200.

One type of monofunctional ethylenically unsaturated monomer that may be particularly suitable is of Formula I, above, wherein R₁ is H or a C₁₋₃₀ alkyl, and R₂ is COOM wherein M is a C₁₋₃₀ straight or branched chain alkyl which may be substituted with one or more hydroxy groups.

More specifically, R₁ is H or CH₃, and R₂ is COOM wherein M is a C₁₋₃₀ straight or branched chain alkyl which may be substituted with one or more hydroxy groups. One specific type of monofunctional ethylenically unsaturated monomer that may be used is a mixture of monomers of Formula I where in one monomer R₁ is H or CH₃ and R₂ is COOM where M is a C₁₋₁₀ alkyl, and where in the second monomer R₁ is H or CH₃, and R₂ is COOM where M is a C₁₋₁₀ alkyl substituted with one or more hydroxy groups.

Di-, tri- and polyfunctional monomers, as well as oligomers, of the above monofunctional monomers may also be used to form the polymer. Suitable difunctional monomers include those having the general formula: 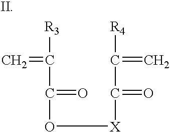
wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas: 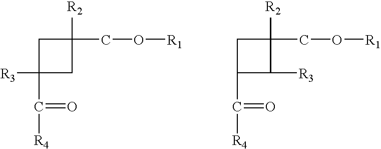
wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane. The monomers mentioned herein can be polymerized with various types of organic groups such as propylene glycol, isocyanates, amides, etc.

One type of organic group that can be polymerized with the above monomers includes a urethane monomer. Urethanes are generally formed by the reaction of polyhydroxy compounds with diisocyanates, as follows: 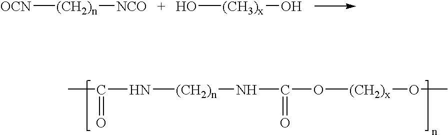
wherein x is 1-1000.

Another type of monomer that may be polymerized with the above comprise amide groups, preferably having the following formula: 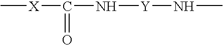
wherein X and Y are each independently linear or branched alkylene having ₁₋₄₀ carbon atoms, which may be substituted with one or more amide, hydrogen, alkyl, aryl, or halogen substituents.

Another type of organic monomer may be alpha or beta pinenes, or terpenes, abietic acid, and the like.

4. Waxes

Also suitable for use as the film forming component are various types of materials commonly referred to as “waxes”. Such waxes may be animal, vegetable, mineral, synthetic, or silicone waxes. Generally such waxes have a melting point ranging from about 28 to 125° C., preferably about 30 to 100° C. Examples of waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on. Other suitable waxes are set forth in U.S. Pat. No. 5,725,845 which is hereby incorporated by reference in its entirety.

5. Higher Molecular Weight Hydrocarbons

Also suitable as the film forming component are one or more higher molecular weight hydrocabons, particular those having a molecular weight of greater than about 500, preferably greater than about 800, more preferably greater than about 1,000. Examples of such hydrocarbons include esters or paraffinic hydrocarbons, lanolin oil or derivatives thereof, and the like.

(a). Esters

Suitable esters include mono-, di-, or triesters. Monoesters are defined as esters formed by the reaction of a monocarboxylic acid having the formula R—COOH, wherein R is a straight or branched chain saturated or unsaturated alkyl having from 1 to 500 carbon atoms, or phenyl; and an alcohol having the formula R—OH wherein R is a straight or branched chain saturated or unsaturated alkyl having 1-500 carbon atoms, or phenyl. Both the alcohol and the acid may be substituted with one or more hydroxyl groups. Either one or both of the acid or alcohol may be a “fatty” acid or alcohol, ie. may have from about 6 to 22 carbon atoms.

Suitable diesters that may be used in the compositions of the invention are the reaction product of a dicarboxylic acid and an aliphatic or aromatic alcohol, or an alcohol having two hydroxyl groups with a carboxylic acid. The mono- or dicarboxylic acid may contain from 5 to 1000 carbon atoms, and may be in the straight or branched chain, saturated or unsaturated form, and may be substituted with one or more hydroxyl groups. The aliphatic or aromatic alcohol may also contain from 1 to 1,000 carbon atoms, and may be in the straight or branched chain, saturated, or unsaturated form. The aliphatic or aromatic alcohol may be substituted with one or more substituents such as hydroxyl. Preferably, one or more of the acid or alcohol is a fatty acid or alcohol, i.e. contains 14-22 carbon atoms.

Suitable triesters comprise the reaction product of a tricarboxylic acid and an aliphatic or aromatic alcohol or an alcohol having three hydroxyl groups with a carboxylic acid. As with the mono- and diesters mentioned above, the acid and alcohol contain from to 1,000 carbon atoms, and may be saturated or unsaturated, straight or branched chain, and may be substituted with one or more hydroxyl groups.

One particularly suitable ester for use in the compositions of the invention is castor oil or derivatives thereof.

(b). Hydrocarbon Oils.

Also suitable film forming components include non-volatile hydrocarbon oils. The term “nonvolatile” means that the oil has a vapor pressure of less than about 2 mm. of mercury at 20° C.

Suitable nonvolatile hydrocarbon oils include isoparaffms and olefins having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, mineral oil, pentahydrosqualene, squalene, squalane, and mixtures thereof.

(c). Lanolin Oil

Also suitable for use as the film forming component is lanolin oil or derivatives thereof containing hydroxyl, alkyl, or acetyl groups, such as hydroxylated lanolin, isobutylated lanolin oil, acetylated lanolin, acetylated lanolin alcohol, and so on.

D. Other Ingredients

A variety of other ingredients may be suitable for use in the compositions, including volatile oils, particulates, humectants, surfactants, and the like.

1. Volatile Oils

Suitable volatile oils include volatile cyclic or linear silicones, volatile paraffinic hydrocarbons and mixtures thereof. If present, such volatile oils may range from about 0.1-95%, preferably about 0.5-85%, more preferably about 1-75% by weight of the total composition. Suitable volatile oils further include:

Cyclic silicones (or cyclomethicones) are of the general formula: 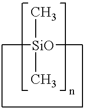
where n=3-6.

Linear volatile silicones in accordance with the invention have the general formula:
(CH₃)₃Si—O—[Si(CH₃)₂—O]ₙ—Si(CH₃)₃
where n=0-7, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids include octamethylcyclotetrasiloxane, decamethylcyclopentasiloxane, dodecamethylcyclohexasiloxane, hexamethyldisiloxane, octamethyltrisiloxane, decamethyltetrasiloxane, and dodecamethylpentasiloxane, and mixtures thereof.

Also suitable as the volatile oils are various straight or branched chain paraffinic hydrocarbons having about 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic hydrocarbons are available from EXXON under the ISOPARS™, and from the Permethyl Corporation. Suitable C₁₂ isoparaffins, such as isododecane, are manufactured by Permethyl Corporation under the tradename Permethyl 99A. Various C₁₆ isoparaffins commercially available, such as isohexadecane (having the tradename Permethyl R), are also suitable.

2. Particulates

In the event the compositions are colored cosmetic compositions, they may contain amounts of particulates ranging from about 0.1-95%, more preferably about 0.5-75% of particulate matter having a particle size of 0.01 to 200, preferably 0.25-100 microns. The particulate matter may be colored or non-colored (for example white) non-pigmentitious powders that may give the cosmetic stick an opaque or semi-opaque quality and contribute to stick structure. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, natural or synthetic fibers, or mixtures thereof. While titanium dioxide is commonly considered to be a white pigment when used in paints, in cosmetics it is used more for its ability to mute color, and/or provide an opaque or semi-opaque finish, then as a colorizing ingredient. The above mentioned powders may be surface treated with lecithin, amino acids, mineral oil, silicone, or various other agents either alone or in combination, which coat the powder surface and render the particles more lipophilic in nature.

The particulate matter component also may comprise various organic and/or inorganic pigments, alone or in admixture with one or more non-pigmentitious powders. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The composition may contain a mixture of both pigmentatious and non-pigmentatious particulate matter. The percentage of pigment used in the particulate matter component will depend on the type of cosmetic being formulated.

3. Surfactants

The compositions of the invention may comprise about 0.01-20%, preferably about 0.1-15%, more preferably about 0.5-10% by weight of the total composition of a surfactant. The surfactant may be nonionic, anionic, cationic, amphoteric, or zwitterionic. The surfactants may be present whether the composition of the invention is in the anhydrous or aqueous emulsion or solution form. If the latter, surfactants are particularly desirable as the facilitate formation of stable emulsions or solutions.

Suitable nonionic surfactants or emulsifiers include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Preferred are alkoxylated alcohols which are formed by the reaction of stearic acid, methyl glucose, and ethoxylated alcohol, otherwise known as PEG-20 methyl glucose sesquiisostearate.

Also suitable as the nonionic surfactant are alkyoxylated carboxylic acids, which are formed by the reaction of a carboxylic acid with an alkylene oxide or with a polymeric ether. The resulting products have the general formula: 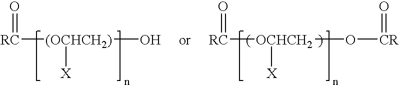
where RCO is the carboxylic ester radical, X is hydrogen or lower alkyl, and n is the number of polymerized alkoxy groups. In the case of the diesters, the two RCO— groups do not need to be identical. The R radical may represent a C₁₋₁₀₀ straight or branched chain, saturated or unsaturated alkyl, but is more preferably a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula: 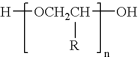
wherein R is H or lower alkyl and n is the number of repeating monomer units, and ranges from 1 to 500.

Other suitable nonionic surfactants include alkoxylated sorbitan and alkoxylated sorb itan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Also suitable as nonionic surfactants are silicone surfactants, which are defined as silicone polymers which have at least one hydrophilic radical and at least one lipophilic radical. Suitable silicone surfactants may be liquid or solid at room temperature, and may be water-in-oil or oil-in-water type surfactants having a Hydrophile/Lipophile Balance (HLB) of 2 to 18. Particularly useful organosiloxane surfactants are nonionic, having an HLB of 2 to 12, preferably 2 to 10, most preferably 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the molecular weight of the lipophilic group.

Examples of silicone surfactants are those sold by Dow Corning under the tradename Dow Corning 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention. Such types of silicone surfactants are generally referred to as dimethicone copolyols or alkyl dimethicone copolyols.

Suitable cationic, anionic, zwitterionic, and amphoteric surfactants are disclosed in U.S. Pat. No. 5,534,265, which is hereby incorporated by reference in its entirety.

4. Sunscreens

If desired, the compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmit UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on pages 1808 to 1809 under the heading “Ultraviolet Light Absorbers” of the CTFA International Cosmetic Ingredient Dictionary and Handbook, Eighth Edition, 2000, as well as U.S. Pat. No. 5,620,965, both of which are hereby incorpated by reference. Examples of such sunscreen materials are p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)]amnobenzoate (ethyl dihydroxypropyl PABA), 2-ethylhexyl-2-cyano-3,3-diphenylacrylate (octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), Padimate O, (Octyl Dimethyl PABA), 2-Phenylbenzimidazole-5-sulfonic acid (Phenylbenzimidazole Sulfonic acid), Red Petrolatum, Sulisobenzone (Benzophenone-4), triethanolamine salicylate (TEA-Salicylates), and so on.

5. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

6. Humectants

If desired, the compositions of the invention may comprise from about 0.01-30%, preferably 0.5-25%, more preferably 1-20% by weight of the total composition of one or more humectants. Suitable humectants include materials glycols, sugars, and similar materials. Suitable glycols include polyethylene and polypropylene glycols such as PEG 4-240, which are polyethylene glycols having from 4 to 240 repeating ethylene oxide units; as well as C₁₋₆ alkylene glycols such as ethylene glycol, propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆ , preferably C₂₋₄ alkylerie glycols, most particularly propylene or butylene glycol.

7. Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, farnesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.

8. Gellants

It may be desireable to include other gallants, either in the oil phase, water, phase, or both phases of the composition to provide gelling. In the case where the composition is anhydrous, the gellant may be included in the oily phase. Such gellants may be included a range of about 0.1-20%, preferably about 1-18%, more preferably about 2-10% by weight of the total composition. Suitable gellants include soaps, i.e. salts of water insoluble fatty acids with various bases. Examples of soaps include the aluminum, calcium, magnesium, potassium, sodium, or zinc salts of C₆₋₃₀, preferably C₁₀₋₂₂ fatty acids.

Also suitable are hydrocolloids such as gellan gum, gum arabic, carrageenan, and those set forth in U.S. Pat. No. 6,197,319 which is hereby incorporated by reference in its entirety.

9. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 1765-1766 of the CTFA International Cosmetic Ingredient Dictionary and Handbook, Eighth Edition, 2000, which is hereby incorporated by reference.

10. Emulsion Stabilizers

If the composition of the invention is in the emulsion form, it may be desirable to incorporate one or more emulsion stabilizers in the composition. If so, suggested ranges are about 0.0001-5%, preferably about 0.0005-3%, more preferably about 0.001-2% by weight of the total composition. Suitable emulsion stabilizers include salts of alkali or alkaline earth metal chlorides or hydroxides, such as sodium chloride, potassium chloride, and the like.

D. Forms of the Cosmetic Composition

The cosmetic compositions of the invention may be found in variety of forms, including mascara, eyeliner, eyeshadow, blush, concealer, foundation makeup, skin brighteners, lipstick, lipliner, lip gloss, powders, lotions, creams, and the like.

1. Foundation Makeup, Color Cosmetics

Foundation makeup or color cosmetics such as eyeshadow, blush, concealer, or eyeliner compositions in the liquid, cream, solid, or stick form. Suitable foundation makeup compositions may be water-in-oil or oil-in-water emulsions. Such compositions generally comprise about:

0.001-85% of the montmorillonite mineral,

0.5-95% water,

0.5-25% particulate matter,

0.01-20% surfactant, and

0.1-95% nonpolar oil.

In addition, these composition may further contain ingredients selected from the group of humectants, preservatives, gellants, film forming polymers, and all of the ingredients as set forth above and in the ranges mentioned. Various anhydrous color cosmetic products may also be suitable, such as blush, powder, lipsticks, eyeshadows, and the like. Such anhydrous color cosmetic compositions may generally comprise about:

0.001-80% of the montmorillonite mineral,

0.1-99% nonpolar oil,

0.1-80% particulate matter; and optionally

0.001-50% wax.

II. The Method

The invention further comprises a method for compatibilizing ingredients in a transfer resistant and/or long wearing cosmetic composition containing at least one film forming component, comprising formulating said composition with at least one non-quaternary montmorillonite mineral.

The montmorillonite material as used in the compositions of the invention is particularly efficacious for stabilizing and compatibilizing the ingredients typically found in such long wearing or transfer resistant cosmetic compositions. The amounts of montmorillonite mineral are as stated in Section I, above. The ingredient particularly provides improvement in undesirable properties such as phase separation, general incompatibility, and the like.

The invention will be further described in connection with the following examples, which are set forth for the purposes of illustration only.

### EXAMPLE 1

Long wearing lipstick compositions were made according to the following formulas:

| | % by weight | | | | |
|---|---|---|---|---|---|
| Ingredient | 1 | 2 | 3 | 4 | 5 |
| Silicone | 57.65 | 59.22 | | 59.22 | 58.92 |
| acrylate | | | | | |
| copolymer¹ | | | | | |
| Trimethylsiloxy | 4.80 | 4.94 | — | 4.94 | 4.91 |
| silicate | | | | | |
| Dimethicone, | 1.96 | 2.01 | 2.12 | 2.01 | 2.00 |
| 60,000 cst. | | | | | |
| Isododecane | 5.82 | 10.07 | 5.51 | 5.24 | 5.21 |
| Mica | 0.88 | — | — | — | — |
| C20-40 | 0.50 | — | — | — | — |
| alcohols | | | | | |
| Magnasperse | 11.73 | 4.83 | 10.17 | 9.66 | 9.62 |
| ISD 1004² | | | | | |
| Pigment grind³ | 17.16 | 17.12 | 18.01 | 17.12 | 17.03 |
| Ethylhexyl | — | 1.81 | 1.91 | 1.81 | 1.80 |
| hydrostearate | | | | | |
| benzoate | | | | | |
| Polyethylene | — | — | — | — | 0.50 |

¹Shin-Etsu KP550
²Monmorillonite clay (26%), isododecane, ethylhexyl stearate, PEG-30 dipolyhydroxystearate, propylene carbonate
³39.7% pigment in isododecane

The compositions were prepared by grinding the pigments and Magnasperse in isododecane. The pigment grind was then combined with the remaining ingredients to form long wearing lipstick compositions.

### EXAMPLE 2

A traditional lipstick formula is prepared as follows:

| Ingredient | % by weight |
|---|---|
| Polyethylene | 9.0 |
| Ceresin wax | 3.0 |
| Ozokerite | 2.0 |
| Trioctyldodecyl citrate | 27.0 |
| Octyldodecyl neopentanoate | 8.0 |
| Caprylic/capric triglycerides | 7.0 |
| Ethylhexyl palmitate | 6.0 |
| C10-30 cholesterol/lanosterol esters | 4.0 |
| Polyisobutene | 9.0 |
| Magnasperse Gel OS (monmorillonite [9.7%], ethyl hexyl | 9.0 |
| stearate, PEG-30 dipolyhydroxystearate, propylene | |
| carbonate) | |
| Retinyl palmitate | 0.1 |
| Tocopheryl acetate | 0.1 |
| Methyl paraben | 0.3 |
| Propyl paraben | 0.1 |
| BHT | 0.1 |
| Sorbic acid | 0.9 |
| D&C Red #7 (33% in trioctyldodecyl citrate) | 2.0 |
| Red iron oxide (50% in trioctyldodecyl citrate) | 9.0 |
| Titanium dioxide (50% in trioctyldodecyl citrate) | 6.0 |
| Black iron oxide (50% in trioctyldodecyl citrate) | 0.8 |
| Mica | 3.1 |
| Mica, titanium dioxide | 0.5 |
| Mica, silica | 1.0 |

The composition is made by grinding the pigments in trioctyldodecyl citrate. Separately, the waxes are melted and combined with the oily ingredients. The pigment grind, particulates, waxes, and oils are combined at elevated temperature and mixed well. The composition is poured into lipstick bullet molds and allowed to cool.

### EXAMPLE 3

A mascara formula is prepared as follows:

| Ingredient | % by weight |
|---|---|
| Dimethicone (1 cst) | 20.50 |
| Isododecane | 29.00 |
| Polysilicone-6 | 20.10 |
| Dibutyl adipate | 2.40 |
| Silica | 4.00 |
| Silica shell | 1.90 |
| Magnasperse Gel ISD 1004 (isododecane, montmorillonite, | 18.00 |
| PEG-30 dipolyhydroxystearate, propylene carbonate) | |
| FD&C Blue #1 Aluminum Lake | 1.40 |
| FD&C Yellow #5 Aluminum Lake | 0.60 |
| D&C Green #5 | 0.05 |
| FD&C Red #40 Aluminum Lake | 1.35 |
| Methyl paraben | 0.30 |
| Propyl paraben | 0.10 |
| Dehydroacetic acid | 0.20 |
| Plant extracts | 0.10 |

The ingredients are combined and mixed well to form a viscous liquid mascara. The mascara is poured into vials.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A stabilized film forming cosmetic composition comprising a stabilizing effective amount of dispersed non-quaternary montmorillonite mineral and a film forming effective amount of a film forming component.

2. The composition of claim 1 wherein the dispersed non-quaternary montmorillonite mineral is a synthetic or natural montmorillonite mineral.

3. The composition of claim 1 wherein the dispersed non-quaternary montmorillonite mineral is a metal silicate.

4. The composition of claim 3 wherein the metal comprises aluminum, magnesium, sodium, potassium, lithium, beryllium, or mixtures thereof.

5. The composition of claim 1 wherein the montmorillonite mineral is treated with one or more dispersion enhancing agents.

6. The composition of claim 5 wherein the dispersion enhancing agents include one or more film forming components.

7. The composition of claim 5 wherein the dispersion enhancing agents are fatty acid esters, polyesters, polyethers, polyester/polyethers, carbonates, or mixtures thereof.

8. The composition of claim 5 wherein the dispersion enhancing agents are esters of C₁₋₂₀ alcohols and one or more fatty acids where one or more of the alcohol or acid are substituted with one or more hydroxyl groups.

9. The composition of claim 5 wherein the dispersion enhancing agent is a polyester or polyether containing repeating glycol ether and fatty ester groups.

10. The composition of claim 5 wherein the dispersion enhancing agents are glycol ether groups are alkylene glycol and the fatty ester groups are C₈₋₂₂ fatty acids.

11. The composition of claim 1 wherein the non-quaternary montmorillonite mineral comprises a metal silicate in combination with at least one dispersion enhancing agent.

12. The composition of claim 11 wherein the metal silicate comprises interconnected platelets.

13. The composition of claim 12 wherein the dispersion enhancing agent separates the interconnected platelets of the non-quaternary montmorillonite mineral.

14. The composition of claim 13 wherein the dispersion enhancing agent comprises a film forming component.

15. The composition of claim 13 wherein the dispersion enhancing agent comprises a fatty acid ester or hydroxy fatty acid ester, or a polyester or polyether.

16. The composition of claim 1 wherein the film forming component is a silicone film forming polymer.

17. The composition of claim 16 wherein the silicone film forming polymer comprises from about 0.01-95% by weight of the total composition.

18. The composition of claim 16 wherein the silicone film forming polymer comprises monofunctional units, difunctional units, trifunctional units, quadrafunctional units, or combinations thereof.

19. The composition of claim 18 wherein the silicone film forming polymer comprises a silicone resin having monofunctional units in combination with trifunctional units or quadrafunctional units.

20. The composition of claim 19 wherein the silicone film forming polymer is trimethylsiloxysilicate, polymethylsilsesquioxane, or mixtures thereof.

21. The composition of claim 17 further comprising from about 0.1-95% by weight of the total composition of particulates.

22. The composition of claim 21 wherein the particulates comprise non-pigmentitious powders, organic pigments, inorganic pigments, or mixtures thereof.

23. The composition of claim 1 wherein the film forming polymer comprises a copolymer of silicone and organic monomers.

24. The composition of claim 23 wherein the silicone monomers are monofunctional, difunctional, trifunctional or quadrafunctional.

25. The composition of claim 24 wherein the organic monomers comprise one or more ethylenically unsaturated monomers.

26. The composition of claim 25 wherein the film forming polymer comprises a copolymer of monofunctional and difunctional siloxane units and the organic monomers comprise one or more ethylenically unsataturated monomers.

27. The composition of claim 25 wherein the film forming polymer comprises a copolymer of monofunctional and quadrafunctional units and the organic monomers comprise one or more ethylenically unsaturated monomers.

28. The composition of claim 25 wherein the film forming polymer comprises a copolymer of monofunctional and trifunctional units and the organic monomers comprise one or more ethylenically unsaturated monomers.

29. The composition of claim 25 further comprising from about 0.1-95% by weight of the total composition of particulates.

30. The composition of claim 29 wherein the particulates comprise non-pigmentitious powders, organic pigments, inorganic pigments, or mixtures thereof.

31. The composition of claim 1 wherein the film forming component comprises a film forming polymer formed from ethylenically unsaturated monomers.

32. The composition of claim 31 further comprising from about 0.1-95% by weight of the total composition of particulates comprising non-pigmentitious powders, organic pigments, inorganic pigments, or mixtures thereof.

33. A transfer resistant lipstick composition comprising a stabilizing effective amount of a non-quaternary montmorillonite mineral and a film forming effective amount of a film forming polymer selected from the group consisting of a silicone polymer, a polymer of silicone and organic groups, a polymer of organic groups, and mixtures thereof.

34. The composition of claim 33 comprising, by weight of the total composition:
about 0.001-85% of the non-quaternary montmorillonite mineral,
about 0.01-95% of the film forming polymer,
about 0.1-95% of one or more volatile oils; and
about 0.1-95% of one or more particulates.

35. The composition of claim 34 wherein the non-quaternary montmorillonite mineral is treated with one or more dispersion enhancing agents.

36. The composition of claim 35 wherein the dispersion enhancing agents are selected from the group consisting of polyesters, polyethers, polyester/polyethers, carbonates, and mixtures thereof.

37. The composition of claim 34 wherein the film forming polymer is a copolymer of silicone and ethylenically unsaturated monomers.

38. The composition of claim 37 wherein the film forming polymer is a copolymer of silicone and acrylic acid, methacrylic acid, or their simple esters.

39. The composition of claim 35 wherein the volatile oils are linear or cyclic volatile silicones or paraffinic hydrocarbons.

40. The composition of claim 35 further comprising one or more esters.

41. A long wearing lipstick composition comprising a stabilizing effective amount of a non-quaternary montmorillonite mineral and at least one film forming component.

42. The composition of claim 41 comprising, by weight of the total composition:
about 0.001-85% of the non-quaternry montmorillonite mineral,
about 0.01-95% of the film forming component,
about 0.1-95% of one or more volatile oils; and
about 0.1-95% of one or more particulates.

43. The composition of claim 42 wherein the non-quaternary montmorillonite mineral is treated with one or more dispersion enhancing agents.

44. The composition of claim 42 wherein the at least one film forming component comprises a high molecular weight hydrocarbon or wax.

45. The composition of claim 44 wherein the high molecular weight hydrocarbon comprises an ester having a molecular weight of greater than about 500.

46. The composition of claim 44 wherein the wax is a hydrocarbon wax or a silicone wax having a melting point ranging from about 30 to 125° C.

47. A stabilized film forming color cosmetic composition comprising a stabilizing effective amount of a dispersed non-quaternary montmorillonite mineral treated with one or more dispersion enhancing agents, in combination with a film forming polymer selected from the group consisting of silicone polymers, copolymers of silicone and organic groups, polymers from ethylenically unsaturated monomers, and mixtures thereof.

48. The composition of claim 47 wherein the composition comprises a lipstick, blush, eyeshadow, foundation makeup, mascara, concealer, or eyeliner.

49. A method for compatibilizing ingredients in long wearing or transfer resistant cosmetic compositions by formulating such compositions with a non-quaternary montmorillonite mineral.

50. The method of claim 49 wherein the non-quaternary montmorillonite mineral is present from about 0.001-95% by weight of the total composition.

51. A cosmetic composition comprising at least one non-quaternary montmorillonite mineral in nanoplatelet form.

52. The composition of claim 51 which is anhydrous.

53. The composition of claim 51 which is an emulsion.

54. The composition of claim 51 which further comprises one or more volatile oils.

55. The composition of claim 51 which is a lipstick.
